(19) United States
(12) Patent Application Publication
(10) Pub. No.: US 2026/0105991 A1
(43) Pub. Date: Apr. 16, 2026

(54) SYSTEM AND METHOD FOR UNIFIED ENZYME ENGINEERING FRAMEWORK

(71) Applicant: Quantiphi, Inc., Marlborough, MA (US)

(72) Inventors: Dagnachew Birru, Marlborough, MA (US); Vishal Pagidipally, Toronto (CA); Tehemton K Khairabadi, Mumbai (IN); Pooja Kesari, Mumbai (IN); Meghana Veeramalla, Mumbai (IN); Anasuya Chatterjee, Mumbai (IN); Swetha CM, Mumbai (IN)

(21) Appl. No.: 19/422,881

(22) Filed: Dec. 17, 2025

(51) Int. Cl. G16B 30/20; G16B 30/10; G16B 35/20

(52) U.S. Cl. CPC G16B 30/20; G16B 30/10; G16B 35/20

(57) ABSTRACT

A method (400) and system (100) to prepare a framework for enzyme optimization is disclosed. The method (400) includes executing a plurality of optimization modules to obtain engineered enzyme variants. The method (400) may include conducting a similarity search against one or more known enzyme databases. The method (400) may further include obtaining a multi-property optimization (MPO) score by evaluating the engineered enzyme variants generated by the optimization modules according to predefined goals. The method (400) may further include generating a feedback based on the MPO score to iteratively refine the optimization modules and improve enzyme variant properties.

### FIELD OF THE INVENTION

The present disclosure relates to chemical compounds, and more specifically to a system and method for engineered framework for optimization of protein sequence.

### BACKGROUND OF THE INVENTION

Enzymes are widely utilized in industrial, pharmaceutical, environmental, and biochemical applications. However, naturally occurring enzymes often exhibit limitations in substrate specificity, cofactor compatibility, catalytic efficiency, metal-ion coordination, thermostability, and immunogenicity. Traditional enzyme engineering techniques, including directed evolution and rational design, require extensive experimental screening and may fail to capture the complex interplay among structural, electrostatic, and allosteric determinants of enzyme function.

Advances in computational biology, protein modelling, and machine learning have enabled in silico analysis and redesign of enzymes at a larger scale. Yet existing computational pipelines generally address only isolated aspects of enzyme performance, such as substrate binding or structural stability, without integrating multiple functional properties into a unified optimization framework. Furthermore, conventional approaches lack iterative feedback mechanisms that refine predictions based on multi-parameter performance constraints.

Additionally, conventional techniques often lack interpretability, operating as black-box systems where the rationale behind sequence selection is not transparent. The lack of explainability hinders rational design and limits the ability to target specific regions or properties of the protein. Furthermore, the conventional approaches struggle to balance exploration of the protein sequence space with exploitation of known high-performing regions, often converging to local optima rather than global solutions.

There remains a need for improved computational systems and methods capable of analysing enzyme scaffolds, predicting structural and functional features, and redesigning enzyme variants using coordinated optimization modules. Such systems should incorporate similarity searches, multi-domain functional assessments, and iterative, score-driven refinement to generate optimized enzyme sequences. The methods and systems described herein provide an integrated computational framework for enzyme optimization, redesign, and evaluation across multiple biochemical performance criteria.

### SUMMARY

The following embodiments presents a simplified summary in order to provide a basic understanding of some aspects of the disclosed invention. This summary is not an extensive overview, and it is not intended to identify key/critical elements or to delineate the scope thereof. Its sole purpose is to present some concepts in a simplified form as a prelude to the more detailed description that is presented later.

The present disclosure provides an integrated computational framework for enzyme optimization that unifies structural analysis, functional characterization, and multi-parameter redesign within an iterative, score-driven architecture. Traditional enzyme engineering approaches typically address individual biochemical properties in isolation or rely heavily on experimental screening, which limits efficiency and scalability. In contrast, the framework described herein employs coordinated optimization modules, similarity-based scaffold characterization, and iterative feedback mechanisms to generate improved enzyme variants that meet multiple predefined performance criteria.

According to some example embodiments, the present disclosure provides a computer-implemented method for preparing a framework for enzyme optimization. The method includes supplying a starting enzyme structure comprising an enzyme scaffold library, performing similarity searches to characterize each scaffold with respect to structural and functional properties, executing a suite of optimization modules to generate engineered enzyme variants, evaluating those variants against predefined criteria to compute a multi-property optimization (MPO) score, generating feedback based on the MPO score, and producing optimized enzyme sequences in accordance with the optimization goals. The optimization modules include cofactor-binding optimization, substrate-binding optimization, metal-ion coordination optimization, hinge interaction optimization, surface and electrostatics optimization, and allosteric network optimization.

According to some example embodiments, the present disclosure provides a computer-implemented system comprising a data input module, a similarity-search module, an optimization module suite, an evaluation module, a feedback-generation module, and an output module. These components cooperatively analyze enzyme scaffolds, implement domain-specific redesign strategies, evaluate variant performance, and iteratively refine redesign parameters. The system may further include a storage database for maintaining scaffold libraries, engineered variants, MPO scores, and optimized sequences.

According to some example embodiments, the present disclosure provides a non-transitory computer-readable medium storing instructions that, when executed by a computing device, cause the device to perform the enzyme optimization method described herein, including scaffold characterization, multi-domain optimization, MPO-based evaluation, iterative refinement, and output of optimized enzyme sequences.

According to some example embodiments, taken together, the methods, systems, and computer-readable media of the present disclosure provide a comprehensive computational architecture that integrates structural biology, enzymology, molecular modelling, and machine learning to generate enzyme variants exhibiting improved functional and biochemical performance across multiple design objectives.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF DRAWINGS

The above and still further example embodiments of the present invention will become apparent upon consideration of the following detailed description of embodiments thereof, especially when taken in conjunction with the accompanying drawings, and wherein:

FIG. 1 is a block diagram of computer-implemented system for preparing a framework for enzyme optimization, in accordance with an example embodiment.

FIG. 2 is a block diagram illustrating various optimization modules within an optimization module suite configured to generate engineered enzyme variants based on the characterized enzyme scaffolds, in accordance with an example embodiment.

FIG. 3 illustrate a flow diagram of a computer-implemented method to prepare a framework for enzyme optimization, in accordance with an example embodiment.

FIG. 4 illustrate a flow diagram of a non-transitory computer-readable medium storing instructions that, when executed by a computing device, cause the computing device to perform a method for preparing a framework for enzyme optimization, in accordance with an example embodiment.

FIG. 5 is a block diagram of an exemplary workflow for a multi-parameter enzyme optimization framework for implementing embodiments consistent with the present disclosure.

FIG. 6 is a block diagram of an exemplary workflow for zinc-coordination pocket analysis, structural prediction, and sequence optimization for implementing embodiments consistent with the present disclosure.

FIG. 7 is a block diagram of an exemplary workflow for characterizing and re-designing a substrate-binding pocket for implementing embodiments consistent with the present disclosure.

FIG. 8 is a block diagram of an exemplary workflow for optimizing a cofactor-binding domain for implementing embodiments consistent with the present disclosure.

FIG. 9 is a block diagram of an exemplary workflow for modulating hinge-movement kinetics in enzymes to influence conformational rate switching and thereby improve catalytic performance for implementing embodiments consistent with the present disclosure.

FIG. 10 is a block diagram of an exemplary workflow for reducing the immunogenicity potential of an engineered enzyme by identifying and redesigning B-cell epitope hotspots for implementing embodiments consistent with the present disclosure.

The figures illustrate embodiments of the invention for purposes of illustration only. One skilled in the art will readily recognize from the following description that alternative embodiments of the structures and methods illustrated herein may be employed without departing from the principles of the invention described herein.

### DETAILED DESCRIPTION

In the following description, for purposes of explanation, numerous specific details are set forth in order to provide a thorough understanding of the present invention. It will be apparent, however, to one skilled in the art that the present invention can be practiced without these specific details. In other instances, systems, apparatuses, and methods are shown in block diagram form only in order to avoid obscuring the present invention. Further, this application hereby incorporates by reference in its entirety the contents of U.S. patent application Ser. No. 19/309,027 filed 25 Aug. 2025.

Reference in this specification to “one embodiment” or “an embodiment” or “example embodiment” means that a particular feature, structure, or characteristic described in connection with the embodiment is included in at least one embodiment of the present invention. The appearance of the phrase “in one embodiment” in various places in the specification are not necessarily all referring to the same embodiment, nor are separate or alternative embodiments mutually exclusive of other embodiments. Further, the terms “a” and “an” herein do not denote a limitation of quantity but rather denote the presence of at least one of the referenced items. Moreover, various features are described which may be exhibited by some embodiments and not by others. Similarly, various requirements are described which may be requirements for some embodiments but not for other embodiments.

Some embodiments of the present disclosure will now be described more fully hereinafter with reference to the accompanying drawings, in which some, but not all, embodiments of the invention are shown. Indeed, various embodiments of the invention may be embodied in many different forms and should not be construed as limited to the embodiments set forth herein; rather, these embodiments are provided so that this disclosure will satisfy applicable legal requirements. Like reference numerals refer to like elements throughout.

The terms “comprise”, “comprising”, “includes”, or any other variations thereof, are intended to cover a non-exclusive inclusion, such that a setup, device, or method that comprises a list of components or steps does not include only those components or steps but may include other components or steps not expressly listed or inherent to such setup or device or method. In other words, one or more elements in a system or apparatus proceeded by “comprises... a” does not, without more constraints, preclude the existence of other elements or additional elements in the system or method.

Furthermore, one or more computer-readable storage media may be utilized in implementing embodiments consistent with the present invention. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., are non-transitory. Examples include random access memory (RAM), read-only memory (ROM), volatile memory, non-volatile memory, hard drives, CD ROMs, DVDs, flash drives, disks, and any other known physical storage media.

The embodiments are described herein for illustrative purposes and are subject to many variations. It is understood that various omissions and substitutions of equivalents are contemplated as circumstances may suggest or render expedient but are intended to cover the application or implementation without departing from the spirit or the scope of the present invention. Further, it is to be understood that the phraseology and terminology employed herein are for the purpose of the description and should not be regarded as limiting. Any heading utilized within this description is for convenience only and has no legal or limiting effect.

### Definitions

The term “enzyme scaffold” may be used to refer to a structural framework of an enzyme that provides the overall three-dimensional architecture necessary to support its function. It typically includes the conserved backbone, folds, domains, and core residues that maintain stability and shape, while allowing certain regions—such as active sites, loops, or binding pockets—to be modified, engineered, or optimized.

The term “multi-property optimization (MPO)” refers to a simultaneous optimization of several enzyme attributes, including but not limited to catalytic efficiency, substrate specificity, thermostability, pH tolerance, folding stability, expression yield, or cofactor compatibility, using computational, experimental, or hybrid strategies.

The term “Protein Sequence” may be used to refer to a linear arrangement of amino acids that defines the primary structure of a protein. The protein sequence may be generated by an inverse folding model based on a given protein backbone structure, represented as a string of amino acid residues, each selected from a set of naturally occurring or modified amino acids.

The term “Amino acid” may refer to an organic molecule that serves as a building block of proteins, characterized by an amino group, a carboxyl group, and a variable side chain that determines its chemical properties. The amino acid is a single residue within a protein sequence, selected from naturally occurring amino acids or modified variants, which is evaluated and optimized for its contribution to a target property, such as thermostability, through computational methods involving sequence generation and analysis.

The term “cofactor binding” may refer to a physical and chemical association of an enzyme with a cofactor—such as a metal ion, nucleotide-derived molecule, organic prosthetic group, or redox-active compound—that is necessary or beneficial for catalytic function, structural stability, or electron transfer. The term includes binding mediated by specific residues, binding pockets, coordination spheres, or induced-fit structural changes.

The term “module” used herein may refer to a hardware processor including a Central Processing Unit (CPU), an Application-Specific Integrated Circuit (ASIC), an Application-Specific Instruction-Set Processor (ASIP), a Graphics Processing Unit (GPU), a Physics Processing Unit (PPU), a Digital Signal Processor (DSP), a Field Programmable Gate Array (FPGA), a Programmable Logic Device (PLD), a Controller, a Microcontroller unit, a Processor, a Microprocessor, an ARM, or the like, or any combination thereof.

### End of Descriptions

The present disclosure provides a comprehensive, modular, and extensible computational framework designed to support systematic enzyme optimization by integrating structural analysis, functional characterization, multi-property redesign, and iterative score-driven refinement. The framework receives one or more starting enzyme structures, analyses their scaffold characteristics, and generates improved variants by applying targeted optimization strategies implemented in dedicated software components. These components operate together to evaluate biochemical, structural, and functional attributes of enzymes and propose modifications that enhance performance across multiple predefined objectives.

The workflow begins by supplying a starting enzyme structure that includes or references an enzyme scaffold library derived from natural proteins, designed scaffolds, metagenomic sequences, ancestral reconstructions, or previously engineered variants. The system performs a similarity search against known enzyme databases to characterize each scaffold based on structural and functional descriptors such as domain composition, active-site architecture, metal coordination geometry, substrate-binding pocket features, immunogenicity profiles, stability parameters, and kinetic properties. Both alignment-based and structure-based comparison algorithms may be used to identify evolutionary analogs, structural homologs, or conserved functional motifs.

Once characterization is complete, the system executes a suite of optimization modules configured to propose residue-level modifications that improve specific properties. These modules analyze structural and energetic contexts and generate engineered variants along distinct design axes. Examples include cofactor-binding optimization modules that identify residues interacting with NAD⁺, NADP⁺, FAD, FMN, heme, or metal-sulfur clusters and propose changes to modulate steric or electrostatic interactions; substrate-binding optimization modules that evaluate pocket geometry and adjust steric or electrostatic constraints to influence specificity or catalytic turnover; metal-ion coordination optimization modules that modify second-shell or distal residues to tune redox potential, Lewis acidity, or substrate positioning; hinge-interaction optimization modules that adjust loop flexibility or conformational dynamics to influence catalytic cycling; surface and electrostatics modules that alter charged or hydrophobic regions to affect stability, solubility, or long-range electrostatics; and allosteric network modules that identify and rewire communication pathways connecting distal nodes to the catalytic center using molecular dynamics, correlated motion analysis, or evolutionary coupling data. Each module operates independently yet contributes results to a shared design space, which allows the system to process, compare, and evaluate variants generated by different optimization strategies.

The framework is not limited to traditional catalytic or structural properties. Because it can integrate any attribute that can be quantitatively or semi-quantitatively scored, the system also supports optimization of pH stability, thermal stability, solubility, aggregation resistance, oxidative robustness, expression level, or post-translational compatibility. The architecture is model-agnostic and scoring-agnostic; scoring functions, simulation engines, or predictive models can be swapped, updated, or replaced without altering the overall workflow. This modularity allows the disclosure to extend beyond enzyme engineering to other protein design domains, including antibody optimization, biosensor tuning, and de novo protein design.

After variant generation, the framework computes a multi-property optimization (MPO) score that aggregates normalized property-specific scores into a single scalar measure. Each property contributes to the MPO score through a weighting factor that reflects its relative importance in the design context. These weights determine how properties are prioritized, balanced, or traded off during optimization and define the degree to which each property can be improved relative to others. Because mutations often exhibit epistatic interactions, the MPO scoring system provides a holistic measure that accounts for interdependencies among catalytic, structural, and stability parameters. By mapping variant performance across multiple dimensions, the system can identify a Pareto-optimal set of variants that represent the best achievable trade-offs across all evaluated properties.

The MPO evaluation triggers a feedback mechanism that updates or recalibrates module parameters. This feedback may shift sampling strategies, adjust structural constraints, refine scoring weights, redistribute search priorities, or guide exploration toward under-sampled sequence regions. Through repeated iterations, the framework converges toward high-performing enzyme variants that satisfy the predefined optimization goals. At the end of one or more optimization cycles, the system outputs optimized enzyme sequences that align with user-defined or algorithmically derived criteria. These sequences may be ranked, clustered, annotated with their predicted structural or functional features, and prepared for experimental validation or downstream computational analyses.

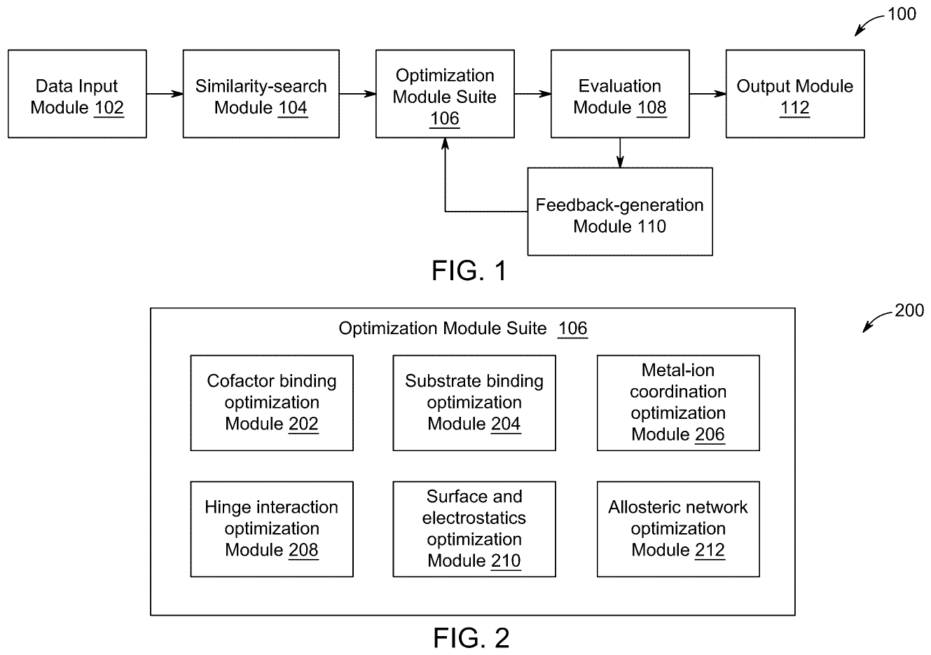

Embodiments of the present disclosure may provide a method, a system, and a non-transitory computer-readable medium storing instructions that, when executed by a computing device, cause the computing device to perform a method for preparing a framework for enzyme optimization. The method, the system, and the a non-transitory computer-readable medium storing instructions are described with reference to FIG. 1 to FIG. 10 as detailed below.

FIG. 1 illustrates an exemplary architecture 100 of a system for enzyme analysis and optimization. As shown, the system 100 includes a data input module 102, which receives or imports the starting enzyme data, such as sequences, structural files, or metadata. The data input module 102 is operatively coupled to a similarity-search module 104, which performs one or more similarity comparisons against external or internal databases to identify related enzyme scaffolds or features.

The output of the similarity-search module 104 is provided to an optimization module suite 106. The optimization module suite 106 may implement one or more enzyme optimization procedures, design strategies, or redesign algorithms based on the initial characterization.

The results from the optimization module suite 106 are communicated to an evaluation module 108, which assesses the generated variants using predefined evaluation metrics or performance criteria. The evaluation module 108 is further coupled to a feedback-generation module 110, which produces feedback signals or updated guidance based on the evaluation results. the feedback-generation module 110 provides the feedback to the optimization module suite 106 to iteratively refine the design process.

Following evaluation, the outputs from the evaluation module 108 are transmitted to an output module 112, which generates the final optimized enzyme sequences, structures, or associated reports.

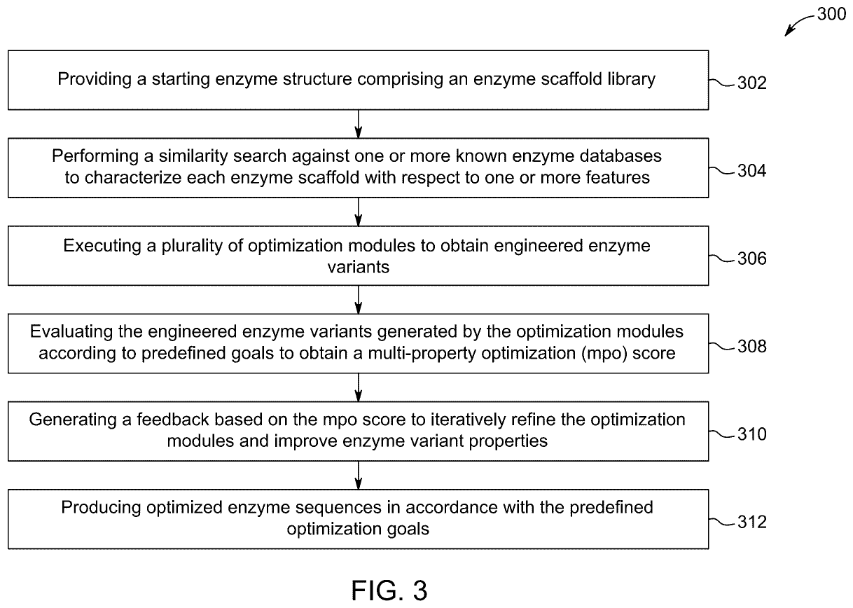

FIG. 2 illustrates an exemplary configuration 200 of the optimization module suite 106. As shown, the optimization module suite 106 includes a plurality of specialized optimization modules, each configured to modify or enhance specific structural or functional aspects of an enzyme. The suite may include a cofactor binding optimization module 202, which is configured to evaluate and redesign residues or pockets involved in cofactor recognition and stabilization. In an embodiment, the framework enables cofactor pocket redesign to modulate cofactor affinity, cofactor usage, and catalytic turnover by reengineering how cofactors such as NAD⁺/NADP⁺, FAD, FMN, heme groups, or metal clusters are positioned and stabilized within the enzyme. This may include modifying residues that directly or indirectly contact the cofactor, adjusting pocket electrostatics or steric constraints to switch between cofactors such as NAD⁺ and NADP⁺, or introducing variations in allosteric regions that influence cofactor binding and release dynamics. Because cofactor affinity and turnover are interdependent—where tighter binding may slow product release—the redesign process incorporates scoring functions that capture both kinetic and structural outcomes. Additionally, in enzymes that rely on conformational transitions such as hinge or lid movements, the redesign must accommodate the dynamic rearrangements required for the cofactor to bind and dissociate efficiently. In more complex systems containing tightly coupled hydride-or electron-transfer pathways, the framework evaluates whether structural perturbations introduced during redesign impact the stability or continuity of the catalytic cycle. This type of targeted modification has been demonstrated, for example, in alcohol dehydrogenases where mutations surrounding the NAD⁺ binding site shift Km values and modulate turnover, and in glucose dehydrogenase where rational residue changes successfully switch the preferred cofactor from NAD⁺ to NADP⁺.

A substrate binding optimization module 204 is provided to improve active-site complementarity, substrate orientation, or catalytic positioning. In an embodiment, the framework can further support substrate-binding pocket engineering to modulate substrate specificity, selectivity, and catalytic efficiency by redesigning how substrates are recognized, oriented, and stabilized within the active site. This includes introducing mutations to residues that line the pocket to either expand its volume for bulkier substrates or restrict access to undesired molecules, while also refining shape complementarity, hydrophobic packing, or electrostatic interactions that favor the intended substrate. In certain implementations, saturation mutagenesis, structure-guided redesign, or computational docking workflows are applied to explore variants that optimize these interactions. Because alterations in this region may affect substrate access channels, binding kinetics, or transition-state stabilization, the system evaluates such variants using property-specific and multi-property scoring functions described elsewhere in this disclosure. This enables systematic engineering of substrate-recognition features across diverse enzyme families, as demonstrated by prior efforts modifying alcohol dehydrogenases to enhance ethanol specificity over methanol and tailoring cytochrome P450 enzymes for improved regio-and stereoselectivity through active-site and tunnel remodeling.

A metal-ion coordination optimization module 206 is included to refine metal-binding sites, coordination geometry, or metal-dependent catalytic features. In an embodiment, the framework supports optimization of metal-ion coordination environments, which play a central role in catalytic function for metalloenzymes that depend on Zn²⁺, Fe²⁺/Fe³⁺, Mn²⁺, Mg²⁺, Cu²⁺, or related cofactors. Metal ions often function as Lewis acids that stabilize negative charge buildup, polarize substrates, or engage in redox cycling, and their coordination typically involves histidine, cysteine, glutamate, or aspartate residues. To modulate catalytic behaviour, the system may introduce mutations in second-shell or distal residues surrounding the metal-binding site, thereby adjusting the geometry, electronic distribution, or solvent accessibility of the coordination sphere while preserving the primary coordinating residues responsible for stable chelation. These modifications can tune Lewis acidity, substrate orientation, redox characteristics, or reaction specificity, and may also include the introduction of new coordination residues to enable accommodation of alternative metal ions, such as replacing a native Fe center with Mn. Because metal-dependent enzymes are highly sensitive to miscoordination, the framework evaluates potential trade-offs—including altered binding affinity or unanticipated shifts in redox potential—and incorporates such factors into the MPO scoring and feedback loops. This approach mirrors well-known biochemical systems, such as carbonic anhydrase, where subtle adjustments to histidine positioning modulate hydration kinetics, or zinc-dependent alcohol dehydrogenase, where local changes in coordinating or proximal residues influence substrate orientation and catalytic turnover.

The system also includes a hinge interaction optimization module 208, which is configured to modify hinge-region flexibility, domain movements, or conformational dynamics of the enzyme scaffold. In an embodiment, the framework supports loop and hinge region engineering, which focuses on tuning conformational dynamics that govern catalytic rates, substrate gating, and product release. Flexible loops that transiently occlude or expose the active site may be mutated to refine their closure timing, adjust local stability, or reshape transient interaction networks, while hinge residues that coordinate larger domain motions can be altered to modulate the angular range and speed of structural transitions. Computational tools such as molecular dynamics simulations are incorporated to identify rate-limiting motions, characterize energy barriers between conformational states, and select residue-level interventions that can shift these motions toward more catalytically productive pathways. Because increasing flexibility can disrupt substrate positioning or compromise overall structural stability, the framework balances entropy and enthalpy contributions to ensure that engineered motions remain within functionally relevant limits. Dynamic effects may also be subtle and become evident only under single-turnover or pre-steady-state conditions, and the scoring components are configured to capture such behavior wherever data or simulations allow. These strategies are consistent with modifications reported in systems such as DNA polymerases, where engineered finger-closing domains improve nucleotide incorporation rates, and CRISPR-associated nucleases like Cas9, where hinge alterations accelerate conformational transitions that follow R-loop formation.

Further, a surface and electrostatics optimization module 210 is provided to adjust surface residues, charge distribution, solvent exposure, or interaction interfaces. In an embodiment, surface and electrostatics engineering is incorporated into the framework to improve stability, solubility, and long-range energetic coupling that influences catalytic behavior. This module enables modifications to surface-exposed residues to introduce stabilizing salt bridges, remove unfavorable hydrophobic patches, or reconfigure charge distributions that modulate the pKa values of catalytic residues deep within the active site. The scoring components evaluate changes in surface entropy and sidechain packing to identify variants that exhibit enhanced thermostability while preserving functional conformations. Because alterations to surface charge networks can propagate through allosteric pathways or disrupt local folding equilibria, the framework incorporates structural and energetic checks to minimize destabilizing effects. These strategies reflect principles demonstrated in systems such as RNase A, where re-optimization of surface charge patterns has been shown to significantly improve thermostability.

An allosteric network optimization module 212 is configured to analyse and redesign long-range communication networks, allosteric switches, or distal residue couplings that influence enzyme function. In an embodiment, allosteric network rewiring is supported within the framework, enabling modulation of enzymatic activity, specificity, and regulatory behaviour through mutation of residues that operate as remote control points rather than direct catalytic participants. The system identifies key allosteric nodes and communication pathways using approaches such as evolutionary coupling analysis, NMR-derived dynamics, and molecular dynamics simulations, allowing the framework to map how perturbations propagate through the protein's energetic landscape. Once these nodes are characterized, the optimization modules explore mutations that strengthen or redirect communication between distal sites and the active site, thereby tuning conformational equilibria and altering functional outputs. Because allosteric mutations often produce nonlinear and context-dependent effects, with distal changes sometimes yielding unexpected shifts in kinetics or substrate preference, the framework incorporates simulation-and data-driven checks to capture such dependencies. These principles are exemplified by enzymes such as phosphofructokinase, where modifications at allosteric control sites significantly influence metabolic regulation and catalytic performance.

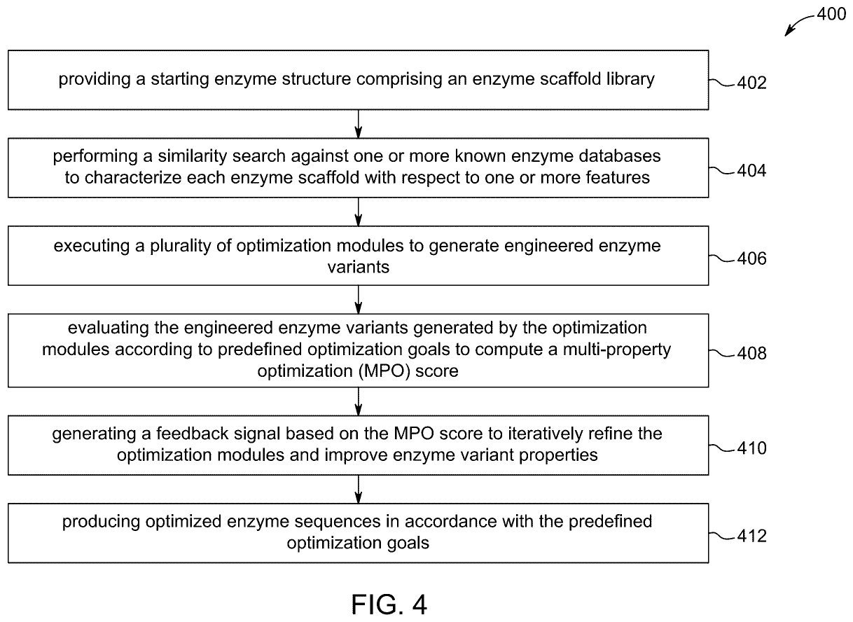

Collectively, the modules within the optimization module suite 106 allow for multifaceted optimization of enzyme scaffolds across structural, chemical, and dynamic dimensions. The complete process followed by the system 100 is explained in detail in conjunction with FIG. 3 to FIG. 10.

FIG. 3 illustrates an exemplary computer-implemented method 300 for preparing a framework for enzyme optimization. As shown, the method 300 begins at step 302, which includes providing a starting enzyme structure comprising an enzyme scaffold library. The starting structure may include sequence data, structural coordinates, or predefined scaffold variants stored in one or more databases.

At step 304, the method includes performing a similarity search against one or more known enzyme databases to characterize each enzyme scaffold with respect to one or more features. Such features may include domains, active-site pockets, chemical environments, cofactor interactions, or other structural and functional characteristics.

At step 306, the method proceeds to executing a plurality of optimization modules to obtain engineered enzyme variants. The optimization modules may correspond to the modules described in FIG. 2, such as cofactor binding optimization, substrate binding optimization, metal-ion coordination optimization, hinge interaction optimization, surface/electrostatics optimization, and allosteric network optimization. In an embodiment, the cofactor-binding optimization module is configured to improve cofactor affinity, enable cofactor switching, or enhance catalytic turnover by optimizing cofactor positioning and binding dynamics. The substrate-binding optimization module is configured to improve substrate specificity, selectivity, or catalytic efficiency by altering substrate recognition and positioning. The metal-ion coordination optimization module is configured to modulate catalytic function by adjusting the metal coordination environment. The hinge interaction optimization module is configured to enhance catalytic turnover (Kcat) by optimizing conformational dynamics involved in substrate binding, catalysis, or product release. The surface and electrostatics optimization module is configured to enhance enzyme stability, solubility, or modulate long-range electrostatic effects on the active site. The allosteric network optimization module is configured to modulate enzyme activity or substrate specificity through reprogramming of remote-control points.

At step 308, the method includes evaluating the engineered enzyme variants generated by the optimization modules according to predefined goals to obtain a multi-property optimization (MPO) score. The evaluation may include assessing stability, activity, binding affinity, structural quality, or other application-specific metrics.

At step 310, the method further includes generating a feedback based on the MPO score to iteratively refine the optimization modules and improve enzyme variant properties. The feedback may be used to adjust parameters, redesign residues, modify constraints, or rerun selected optimization operations.

At step 312, the method concludes by producing optimized enzyme sequences in accordance with the predefined optimization goals, thereby providing output sequences or models ready for downstream analysis, synthesis, or validation.

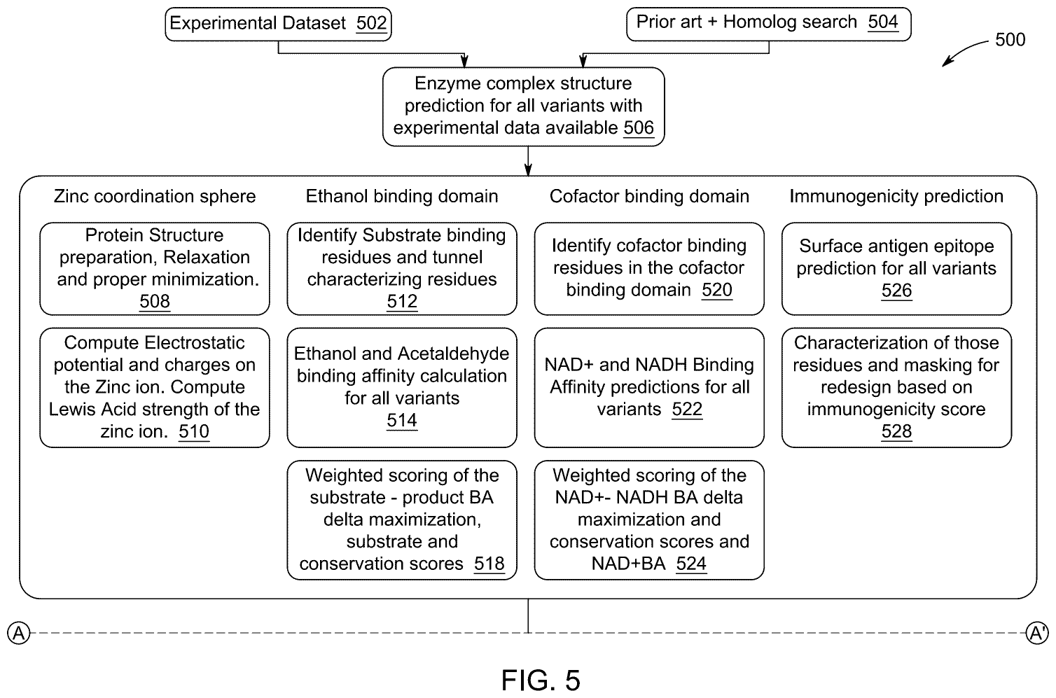

FIG. 4 illustrates an exemplary method 400 encoded on a non-transitory computer-readable medium storing instructions that, when executed by one or more processors of a computing device, cause the computing device to perform a method for preparing a framework for enzyme optimization.

As shown, the method 400 begins at step 402, which includes providing a starting enzyme structure comprising an enzyme scaffold library. The instructions cause the processor to access or retrieve one or more enzyme scaffolds stored in local or remote databases.

At step 404, the instructions cause the processor to perform a similarity search against one or more known enzyme databases to characterize each enzyme scaffold with respect to one or more features. These features may include structural domains, substrate-binding pockets, coordination environments, cofactor interactions, or other biochemical or biophysical properties.

At step 406, the non-transitory medium stores instructions that, when executed, cause the processor to execute a plurality of optimization modules to generate engineered enzyme variants. The optimization modules may include cofactor-binding optimization, substrate-binding optimization, metal-ion coordination optimization, hinge-interaction optimization, surface/electrostatics optimization, and allosteric-network optimization.

At step 408, the stored instructions cause the processor to evaluate the engineered enzyme variants produced by the optimization modules according to predefined optimization goals to compute a multi-property optimization (MPO) score. Evaluation may involve stability scoring, catalytic performance assessments, or structural quality metrics.

At step 410, the medium stores instructions that cause the processor to generate a feedback signal based on the MPO score to iteratively refine the optimization modules and improve enzyme variant properties. This feedback enables repeated cycles of optimization until desired properties are achieved.

Finally, at step 412, the instructions cause the processor to produce optimized enzyme sequences in accordance with the predefined optimization goals, thereby generating final optimized variants suitable for downstream use, validation, or synthesis.

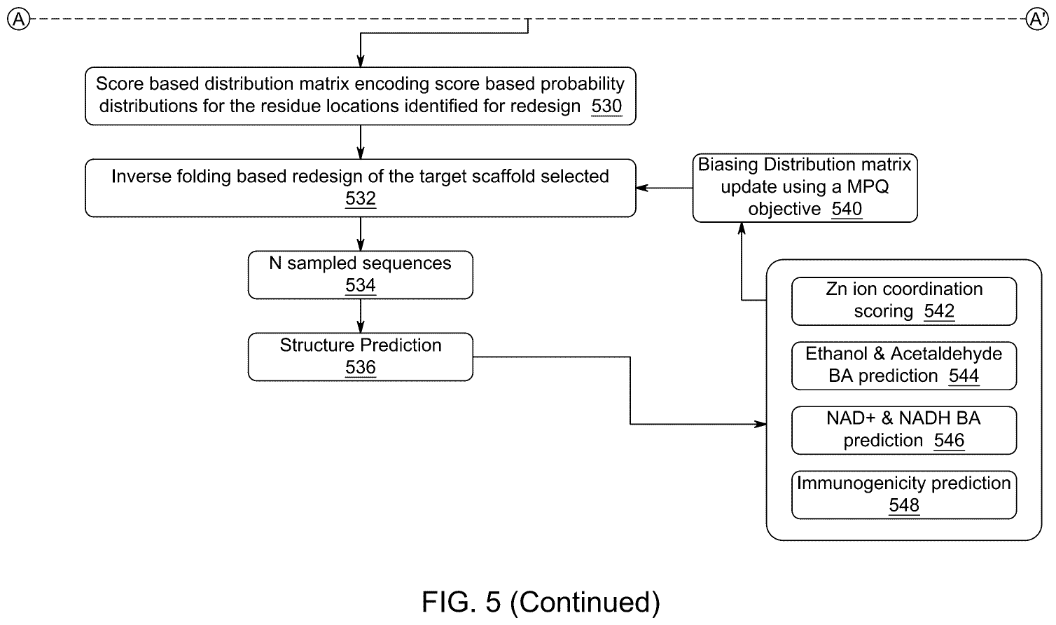

FIG. 5 illustrates an example workflow 500 for a multi-parameter enzyme optimization framework. The workflow begins with the ingestion of an experimental dataset 502 along with information derived from prior art and homolog searches 504, which collectively inform an enzyme complex structure prediction step 506 executed for all variants for which experimental reference data are available. These predicted structures are then analyzed across four functional assessment modules. In certain embodiments, the prior art and homolog searches 504 include identifying structural and familial homologs of the target enzyme and performing multiple-sequence alignment (MSA) across the identified homolog sets. Such alignment procedures can reveal regions of high evolutionary conservation and provide domain-level signals associated with specific protein families. These conservation profiles may be correlated with experimentally determined properties, including catalytic activity, substrate preferences, and domain-specific functional metrics obtained from wet-lab experiments or literature sources, thereby informing which residues or regions should be retained, redesigned, or selectively perturbed. Leveraging evolutionary information obtained from homolog and MSA analyses can accelerate the optimization workflow by guiding residue selection and biasing design operations toward mutational space consistent with functional and structural constraints. The sequence-design component may employ any suitable inverse-folding model, including but not limited to ProteinMPNN or LigandMPNN, which can be incorporated within explainable AI or correlation-and causation-based scoring frameworks to direct optimization toward sequence variants exhibiting improved multi-property performance.

In the zinc-coordination sphere module, the system performs protein structure preparation, relaxation, and minimization 508, followed by computation of electrostatic potential and zinc-ion charge states to determine Lewis acid strength (510). In the ethanol-binding domain module, the workflow identifies substrate-binding and tunnel residues (512), computes ethanol and acetaldehyde binding affinities for all variants (514) and generates weighted substrate scores based on binding-affinity deltas, conservation scores, and product-relevant metrics (518).

In parallel, the cofactor-binding domain module identifies residues involved in cofactor interaction (520) and predicts binding affinities for NAD⁺ and NADH across enzyme variants (522). These outputs are integrated into weighted scoring of NAD⁺/NADH binding-affinity deltas, conservation scores, and cofactor-interaction metrics (524). The immunogenicity module executes surface-epitope prediction for all variants (526) and characterizes residues for redesign-masking based on their immunogenicity scores (528).

The consolidated outputs feed into a score-based distribution-matrix encoding step 530, which generates probability distributions for residue locations selected for redesign. Using these distributions, an inverse-folding-based redesign of a selected target scaffold 532 is performed. A biasing-distribution update 540, computed using a multi-property optimization (MPO) objective, modulates this redesign loop. In an embodiment, the framework may be capable of addressing multiple and potentially competing objectives in enzyme engineering, including improvements in selectivity, substrate affinity, catalytic activity, and other relevant biochemical properties. The framework may incorporate explainable-AI-based optimization techniques in which attribution methods, such as integrated-gradient-based analyses, and correlation-derived signals are utilized to guide multi-objective optimization across diverse structural and functional domains. The distribution matrix may be informed by physics-based simulation outputs in conjunction with AI-driven sampling or generative sequence models, allowing the system to infer an implicit energetic landscape and mutational strategy across domains such as the substrate-binding domain, metal-ion coordination domain, cofactor-binding domain, and domains or loops associated with conformational switching kinetics. The framework may additionally account for further properties, including pH stability, solubility, thermal stability, or other biophysical characteristics for which a scoring function can be defined. As long as a property can be scored and variants can be ranked, the system may optimize for that property. The architecture may be implemented as a modular collection of loosely coupled scoring functions and computational components that may be replaced or updated as simulation methods, biophysical modelling tools, and protein-scoring technologies advance. This model-agnostic and scoring-agnostic configuration allows the same optimization framework to be applied beyond enzyme engineering to other areas of protein design.

In another embodiment, multi-property optimization (MPO) scoring may be employed to generate a scalarized score comprising normalized individual property scores combined as weighted components. The assigned weights may define the relative importance of each property and the extent to which each may be optimized when accounting for epistatic interactions arising from mutations in one domain that affect function in another. The MPO score and its weighting scheme may further determine the Pareto-optimal front when plotting variant properties after scoring, thereby enabling identification of sequence designs that achieve balanced improvements across the full set of targeted biochemical metrics.

Following redesign, the system produces N sampled sequences 534, which undergo structure prediction 536. These redesigned sequences are subsequently evaluated through a downstream scoring module that includes zinc-ion coordination scoring 542, ethanol and acetaldehyde binding-affinity prediction 544, NAD⁺/NADH binding-affinity prediction 546, and immunogenicity prediction 548, thereby enabling iterative refinement of the optimization pipeline. In certain embodiments, enzyme optimization may be conducted through a variety of well-established protein engineering strategies. These may include maintaining key catalytic or structural residues, such as those involved in zinc coordination, to preserve the overall fold and essential metal-binding geometry. Additional modifications may involve introducing mutations that increase the Lewis acidity of the zinc centre or enhance the ability of the catalytic environment to activate the hydroxy functional group of ethanol. Further strategies may include improving local residue packing, modulating electrostatic interactions, or stabilizing the active-site architecture to promote more efficient substrate binding and turnover. Such optimization methodologies are not limited to any single technique and may employ rational design, directed evolution, or other recognized protein engineering tools, individually or in combination.

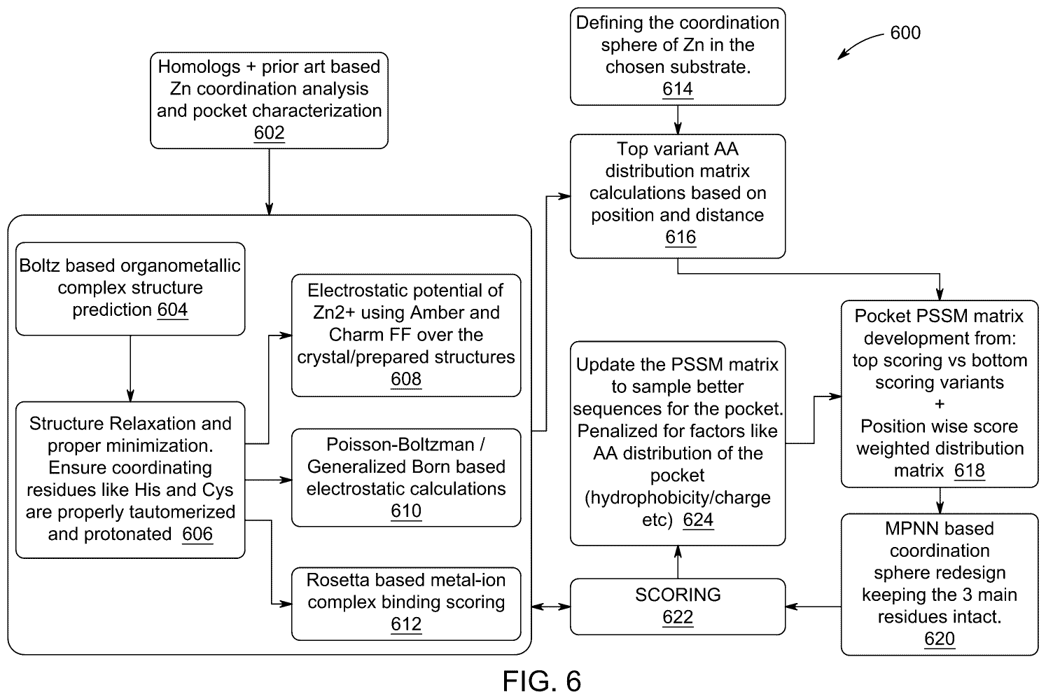

FIG. 6 illustrates an exemplary workflow (600) for zinc-coordination pocket analysis, structural prediction, and sequence optimization. The process begins with homologs and prior-art-based Zn coordination analysis and pocket characterization (602). Output from step 602 is provided to an AlphaFold like co-folding model-based organometallic complex structure prediction (604). The predicted structure then proceeds to structure relaxation and proper minimization (606), in which coordinating residues such as histidine and cysteine are appropriately tautomerized and protonated.

Electrostatic effects are evaluated by calculating the electrostatic potential of Zn²⁺ using Amber and Charmm force fields over the crystal or prepared structures (608). Additional electrostatic refinement is performed using Poisson-Boltzmann/Generalized Born-based electrostatic calculations (610). Metal-ion binding stability is further analyzed through Rosetta-based metal-ion complex binding scoring (612).

Parallel to these steps, the method includes defining the coordination sphere of Zn in the chosen substrate (614). Based on these structural and positional features, the system performs top variant amino-acid (AA) distribution matrix calculations based on position and distance (616).

The results of step 616 feed into pocket PSSM matrix development (618), which is based on: 1) top-scoring versus bottom-scoring variants; and 2) a position-wise score-weighted distribution matrix. In an embodiment, the position-specific scoring matrix (PSSM) is a computational representation that assigns a quantitative score to each amino acid at each position in a protein sequence based on structural, functional, or evolutionary considerations. The matrix reflects how favourable or unfavourable particular substitutions are at defined positions, considering features such as residue frequency, physicochemical compatibility, and predicted effects on stability or activity. In protein engineering, a PSSM is commonly used to guide residue selection, constrain allowable substitutions, and prioritize variants that maintain or improve functional performance. The scoring framework can integrate diverse inputs—including structural constraints, residue-residue interactions, and pocket-specific requirements—to enable systematic, position-wise evaluation of sequence modifications. In the workflow illustrated in FIG. 6, the PSSM is constructed after evaluating a series of structural and electrostatic parameters associated with the zinc coordination environment.

The PSSM information is then used for MPNN-based coordination sphere redesign while keeping the three main residues intact (620). All structural, electrostatic, and scoring data feed into a central scoring module (622). Using the scoring outputs, the system updates the PSSM matrix (624) to sample improved amino-acid sequences for the pocket, applying penalties for factors such as hydrophobicity, charge, and other amino-acid distribution characteristics.

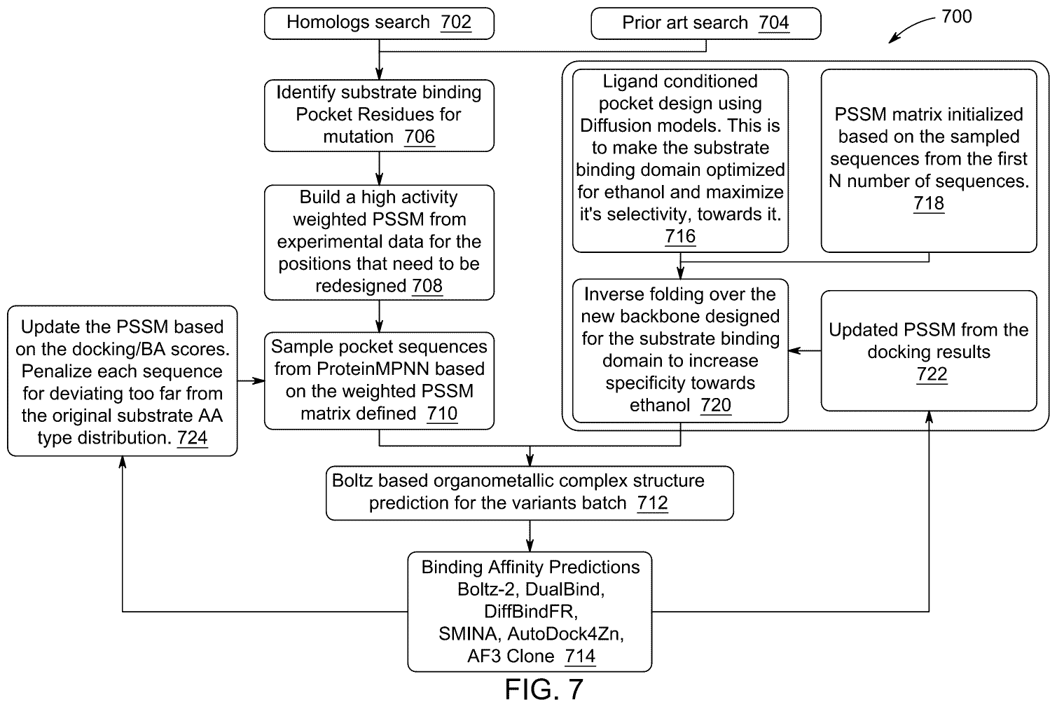

FIG. 7 illustrates an exemplary workflow (700) for characterizing and re-designing a substrate-binding pocket. The substrate-binding pocket may be targeted for various engineering objectives, including improving substrate selectivity, increasing specificity toward a particular substrate molecule, enhancing substrate binding affinity, or reducing Km.

The workflow begins with two inputs: homologs search (702) and prior-art search (704). Information from these searches is used to identify substrate-binding pocket residues for mutation (706). Using these identified positions, the system next performs construction of a high-activity weighted position-specific scoring matrix (PSSM) from experimental data (708) for residues that require redesign. Based on the weighted PSSM, the system samples pocket sequences from ProteinMPNN (710).

The sampled sequences are then supplied to a co-folding model-based organometallic complex structure prediction module for the variants batch (712). The resulting structural models undergo binding affinity prediction (714) using one or more scoring or docking engines, including Boltz-Z, DualBind, DiffBindFR, SMINA, AutoDock4Zn, and AlphaFold based opensource variants.

In parallel, the workflow includes ligand-conditioned pocket design using diffusion models (716) to optimize the substrate-binding domain for ethanol and to maximize selectivity toward the target ligand. The output from step 716 initializes a PSSM matrix based on the first N sampled sequences (718). Backbone redesign of the substrate-binding domain is then performed through inverse folding over the newly designed backbone (720) to further increase specificity toward ethanol.

Docking results are then used to produce an updated PSSM (722), which feeds into an additional refinement step to update the PSSM based on docking and binding-affinity scores (724). In this step, each sequence may be penalized for deviating excessively from the original substrate amino-acid distribution. Outputs from steps 724 and 722 feed back into the sequence-generation and scoring loop, enabling iterative improvement of substrate-binding pocket variants.

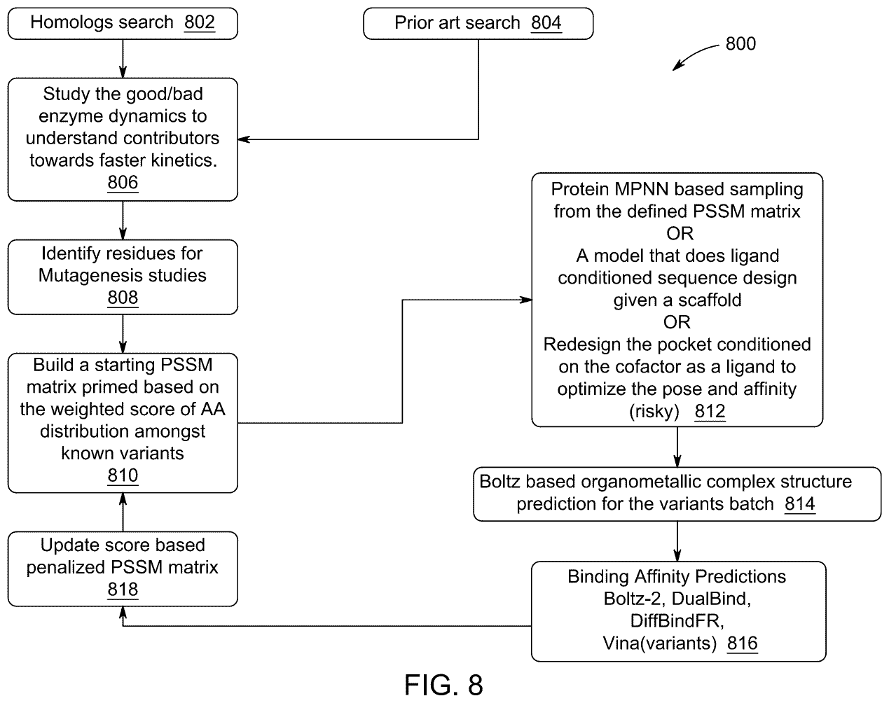

FIG. 8 illustrates an exemplary workflow (800) for optimizing a cofactor-binding domain. Such optimization may be performed to improve cofactor binding affinity, enhance pocket selectivity for the desired cofactor, or refine cofactor orientation and bond geometry to enable more efficient hydride transfer. The workflow begins with a homologs search (802) and a prior-art search (804). Insights from these searches guide the next step, in which the system studies good and bad enzyme dynamics to understand contributors to faster kinetics (806).

Based on the kinetic and structural insights, the method proceeds to identify residues for mutagenesis studies (808). These identified positions are then used to build a starting position-specific scoring matrix (PSSM) (810), which is primed according to weighted amino-acid distributions observed among known variants. The generated PSSM can then be used in one or more advanced sequence-generation routes, represented collectively as module (812), including: ProteinMPNN-based sampling from the defined PSSM matrix, a model performing ligand-conditioned sequence design using a structural scaffold, or pocket redesign conditioned on the cofactor as a ligand to optimize pose and affinity, which may be considered higher risk.

Variants generated through module (812) proceed to an AlphaFold like co-folding model-based organometallic complex structure prediction (814) for the full variant batch. The predicted structures are then evaluated through binding affinity prediction (816) using one or more scoring or docking approaches such as Boltz-Z, DualBind, DiffBindFR, or Vina (variants). Results from step 816 inform an updated score-based penalized PSSM matrix (818), which is fed back into the earlier PSSM-based design loop to iteratively refine and improve cofactor-binding domain variants.

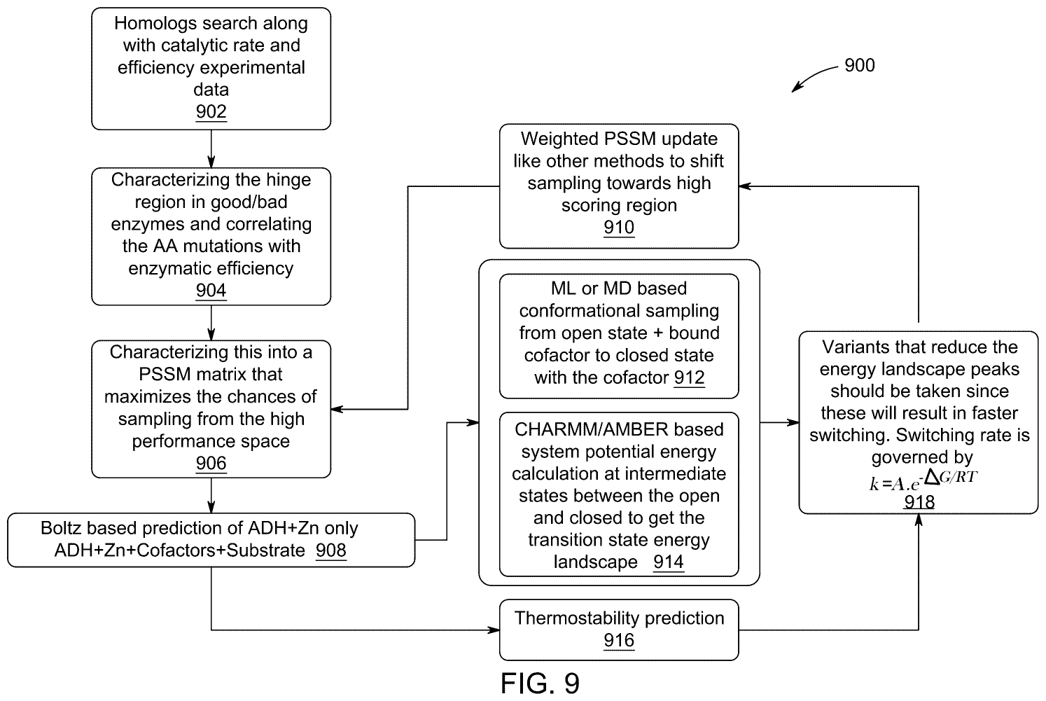

FIG. 9 (900) illustrates an exemplary workflow for modulating hinge-movement kinetics in enzymes to influence conformational rate switching and thereby improve catalytic performance. The figure outlines an integrated computational and experimental framework combining homolog analysis, positional scoring, conformational sampling, energy-landscape evaluation, and thermostability prediction. Modulating hinge-movement kinetics and loop flexibility in enzymes can be useful because these structural elements frequently participate in conformational transitions associated with catalytic turnover. In many enzymatic systems, motions such as loop closure, hinge bending, and substrate-or product-gating influence the effective kcat by regulating access to the active site and stabilizing transient states during catalysis. Thus, engineering loop and hinge regions may provide a means to enhance catalytic efficiency by refining the timing and magnitude of such conformational changes. These modifications, however, involve inherent trade-offs: excess flexibility can impair substrate recognition or reduce overall structural stability, and adjusting motion profiles requires balancing both entropic and enthalpic contributions to the relevant transition states. Additionally, the engineering of these regions presents complexity due to the difficulty of predicting dynamic behaviour, as the resulting functional effects may become apparent only under single-turnover or pre-steady-state kinetic conditions.

The process begins with a homolog search (902), which incorporates catalytic rate and enzymatic efficiency experimental data obtained from related enzymes. The results of this search feed into characterization of the hinge region (904), where hinge-region residues in high-performing and low-performing enzymes are compared, and amino-acid mutations are correlated with observed differences in enzymatic efficiency.

Next, the system generates a PSSM matrix (Position-Specific Scoring Matrix) (906), designed to maximize the probability of sampling sequence variations that fall within a high-performance mutational space. The PSSM is further refined through a weighted PSSM update (910), or equivalent computational methods, to shift sampling toward higher-scoring sequence regions. The PSSM informs Boltzmann-based prediction of enzyme conformational states (908), including ADH+Zn only and ADH+Zn+Cofactor+Substrate combinations. These structural predictions are processed through multiple advanced conformational-modeling pathways.

One such pathway includes machine learning (ML) or molecular dynamics (MD)-based conformational sampling (912), which simulates movements from the open state, through cofactor binding, to the closed state with the cofactor. Another pathway uses CHARMM/AMBER force-field calculations (914) to compute system-level potential energies at intermediate conformational states. These calculations allow estimation of transition-state energy landscapes that govern hinge-movement kinetics.

The output of these analyses identifies sequence variants predicted to lower energy-landscape peaks. As summarized in box (918), variants that reduce energy barriers are preferred because they promote faster conformational switching. The switching rate is governed by the Arrhenius-like relationship: k=A·e^{-ΔG/RT}

Additionally, thermostability prediction (916) is applied to ensure that proposed variants retain or improve stability while enabling enhanced kinetics. Collectively, FIG. 9 (900) presents a comprehensive design scheme for optimizing hinge-movement kinetics in enzymes by integrating structural prediction, energy-landscape modeling, machine learning, and mutational scoring to identify variants capable of improved catalytic switching rates.

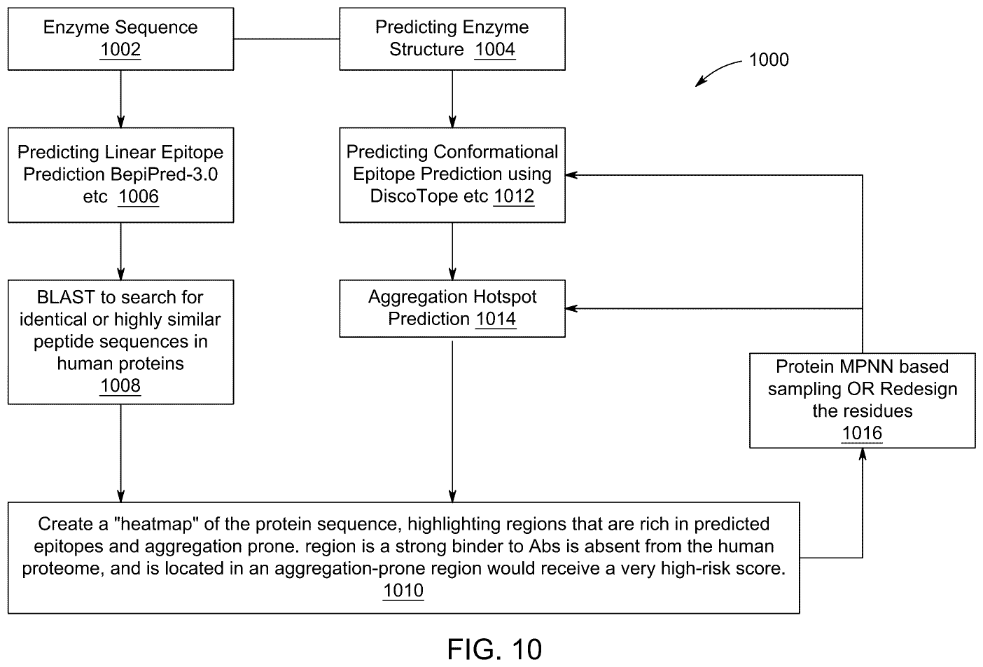

FIG. 10 (1000) illustrates an exemplary workflow for reducing the immunogenicity potential of an engineered enzyme by identifying and redesigning B-cell epitope hotspots. The figure depicts a sequence of computational analyses that characterize immunogenic surface regions, evaluate their similarity to human peptides, assess aggregation propensity, and subsequently redesign high-risk residues using protein-sequence optimization models.

The process begins with provision of the enzyme sequence (1002). In parallel, the corresponding enzyme structure (1004) is predicted or otherwise obtained. Using the sequence information, the system performs linear B-cell epitope prediction (1006), for example using BepiPred-3.0 or similar algorithms. The predicted linear epitopes are then subjected to a BLAST search (1008) to identify identical or highly similar peptide sequences within the human proteome, enabling assessment of potential cross-reactivity. In an embodiment, a BLAST (Basic Local Alignment Search Tool) search for identical or highly similar peptide sequences in human proteins is used to assess whether any region of a candidate enzyme sequence exhibits significant similarity to naturally occurring human peptide segments. In the context of immunogenicity assessment, this type of sequence-comparison step helps identify potential regions that may be recognized as foreign or immunogenic if expressed in vivo. By scanning the redesigned or engineered enzyme sequence against curated human protein databases, the method can flag peptide segments with high sequence identity or conserved motifs that may correlate with immunogenic hotspots. In addition, the immunogenicity-screening workflow may incorporate a B-cell epitope prediction model configured to evaluate the likelihood that specific surface-exposed residues or peptide motifs will trigger an immune response. Although model-training is outside the scope of the invention, the method can utilize such a predictive model to characterize B-cell immunogenicity hotspots and identify positions suitable for redesign. These hotspots may then be selectively modified to reduce their immunogenic potential. Similar to previously disclosed explainable-AI optimization frameworks, the redesign process can employ correlation-and causation-based attribution signals to confine mutational changes to designated surface epitopes, while maintaining the structural and functional integrity of the remaining regions of the enzyme.

In parallel, the predicted or known enzyme structure (1004) is evaluated using conformational epitope prediction methods (1012), such as DiscoTope or related structural immunogenicity models. These conformational predictions, as well as the sequence-based epitope predictions, feed into an aggregation hotspot prediction module (1014), which identifies regions with elevated aggregation propensity that may enhance immunogenic risk.

Based on the linear epitope data, structural epitope predictions, BLAST similarity findings, and aggregation-hotspot analysis, the system generates a risk “heatmap” of the protein (1010). This heatmap highlights sequence regions that are simultaneously enriched for predicted epitopes, show similarity to human-absent antigenic motifs, and/or lie within aggregation-prone segments. Regions that are strong binders to antibodies, absent from the human proteome, and co-localized with aggregation-prone hotspots receive the highest risk scores.

Residues within these high-risk epitope regions are then directed to a Protein MPNN-based sampling or redesign module (1016), which selectively modifies surface-exposed antigenic residues to reduce immunogenic potential. The redesign process is informed by an immunogenicity-prediction model (model training outside the scope of the figure) and uses causal and correlation-based interpretability signals to restrict redesign to immunogenic hotspots while preserving global protein structure and function. Overall, FIG. 10 (1000) provides a computational framework enabling targeted reduction of enzyme immunogenicity through predictive identification and redesign of surface B-cell epitopes.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

It is to be understood that the above description is intended to be illustrative, and not restrictive. For example, the above-discussed embodiments may be used in combination with each other. Many other embodiments will be apparent to those of skill in the art upon reviewing the above description.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

The benefits and advantages which may be provided by the present invention have been described above with regard to specific embodiments. These benefits and advantages, and any elements or limitations that may cause them to occur or to become more pronounced are not to be construed as critical, required, or essential features of any or all of the embodiments.

While the present invention has been described with reference to particular embodiments, it should be understood that the embodiments are illustrative and that the scope of the invention is not limited to these embodiments. Many variations, modifications, additions, and improvements to the embodiments described above are possible. It is contemplated that these variations, modifications, additions, and improvements fall within the scope of the invention.

We claim:

1. A computer-implemented method to prepare a framework for enzyme optimization, the method comprising:
providing a starting enzyme structure comprising an enzyme scaffold library;
performing a similarity search against one or more known enzyme databases to characterize each enzyme scaffold with respect to one or more features selected from the group consisting of enzyme domains, active-site pockets, coordination spheres, immunogenicity profiles, stability, and kinetic parameters;
executing a plurality of optimization modules to obtain engineered enzyme variants;
evaluating the engineered enzyme variants generated by the optimization modules according to predefined goals to obtain a multi-property optimization (MPO) score;
generating a feedback based on the MPO score to iteratively refine the optimization modules and improve enzyme variant properties; and
producing optimized enzyme sequences in accordance with the predefined optimization goals.

2. The computer-implemented method of claim 1, wherein the plurality of optimization modules comprising:
a. cofactor binding optimization;
b. substrate binding optimization;
c. metal ion coordination optimization;
d. hinge interaction optimization;
e. surface and electrostatics optimization; and
f. allosteric network optimization.

3. The computer-implemented method of claim 2, wherein the cofactor-binding optimization module is configured to improve cofactor affinity, enable cofactor switching, or enhance catalytic turnover by optimizing cofactor positioning and binding dynamics, the method comprising:
identifying amino acid residues interacting with one or more cofactors selected from the group consisting of NAD³⁰ , NADP⁺, FAD, FMN, heme, or metal-sulfur clusters;
mutating one or more of said residues to alter steric or electrostatic interactions within the cofactor-binding pocket, thereby modulating binding strength, redox potential, or orientation of the cofactor relative to the catalytic center; and
introducing amino acid substitutions or charge modifications that enable switching between cofactors having distinct charge or size characteristics, including switching between NAD⁺ and NADP⁺ utilization.

4. The computer-implemented method of claim 3, further comprising engineering distal or
allosteric residues to enhance cofactor binding affinity or influence catalytic turnover rate (Kcat) through conformational coupling

5. The computer-implemented method of claim 2, wherein the substrate-binding optimization module is configured to improve substrate specificity, selectivity, or catalytic efficiency by altering substrate recognition and positioning, the method comprising:
identifying residues lining a substrate-binding pocket of the enzyme and characterizing their spatial orientation, hydrophobicity, and electrostatic properties;
mutating one or more residues within the binding pocket to adjust steric constraints, thereby enabling accommodation of bulkier substrates or exclusion of off-target molecules;
optimizing the three-dimensional geometry and electrostatic potential of the pocket to achieve enhanced shape complementarity and charge compatibility with a target substrate; and
employing saturation mutagenesis, molecular docking, or computational screening to identify and select pocket variants exhibiting improved substrate binding affinity or catalytic turnover relative to the parent enzyme.

6. The computer-implemented method of claim 2, wherein the metal-ion coordination optimization module is configured to modulate catalytic function by adjusting the metal coordination environment, the method comprising:
identifying a metal coordination site within the enzyme scaffold and characterizing its geometry, electronic structure, and coordination number;
mutating one or more second-shell or distal residues surrounding the metal-binding site to modify the geometry or electronic environment of the coordination sphere, thereby tuning the Lewis acidity, substrate orientation, redox potential, or reaction specificity; and
maintaining the primary coordinating residues responsible for metal chelation while modifying the electrostatic microenvironment or solvent-access channels to influence metal-ligand interactions.

7. The computer-implemented method of claim 6, further comprising introducing additional coordinating residues or substituting existing residues to enable binding of alternative metal cofactors, thereby allowing replacement of a native metal ion with a different catalytic metal species selected from the group consisting of Fe, Mn, Co, Zn, Cu, and Ni.

8. The computer-implemented method of claim 2, wherein the hinge interaction optimization module is configured to enhance catalytic turnover (Kcat) by optimizing conformational dynamics involved in substrate binding, catalysis, or product release, the method comprising:
identifying flexible loop or hinge regions contributing to active-site occlusion, substrate channeling, or product egress through molecular dynamics simulation or normal mode analysis;
mutating amino acid residues within said loop or hinge regions to modulate conformational flexibility, domain motion, or closure kinetics associated with catalytic turnover; and
adjusting hinge angles or inter-domain linkers to optimize the range or frequency of catalytic conformational transitions.

9. The computer-implemented method of claim 8, further comprising introducing stabilizing or destabilizing substitutions that fine-tune dynamic equilibrium between open and closed enzyme states to reduce rate-limiting conformational barriers.

10. The computer-implemented method of claim 2, wherein the surface and electrostatics optimization module is configured to enhance enzyme stability, solubility, or modulate long-range electrostatic effects on the active site, the method comprising:
identifying surface-exposed residues contributing to hydrophobic patches, charge imbalance, or electrostatic interactions affecting catalytic performance;
introducing one or more charged or polar residues to form stabilizing salt bridges or hydrogen-bond networks that improve overall protein solubility or thermostability; removing surface-exposed hydrophobic residues to reduce aggregation propensity and improve folding efficiency; and
modifying surface charge distribution to adjust the pKa values of active-site residues through long-range electrostatic coupling.

11. The computer-implemented method of claim 10, further comprising introducing mutations that increase surface entropy to enhance thermal stability without perturbing the enzyme's active conformation

12. The computer-implemented method of claim 2, wherein the allosteric network optimization module is configured to modulate enzyme activity or substrate specificity through reprogramming of remote control points, the method comprising:
identifying allosteric nodes or communication pathways within the enzyme using one or more analyses selected from evolutionary coupling analysis, nuclear magnetic resonance (NMR) spectroscopy, molecular dynamics simulation, or correlated motion mapping;
determining residue-residue interaction networks that transmit conformational or energetic signals between distal sites and the catalytic center; and
introducing amino acid substitutions, deletions, or insertions at allosteric nodes to rewire communication pathways and enhance cooperative or inhibitory coupling between functional domains.

13. The computer-implemented method of claim 12, further comprising attenuating inter-domain or intra-domain connectivity to achieve desired modulation of catalytic efficiency, substrate selectivity, or regulatory response.

14. A computer-implemented system for preparing a framework for enzyme optimization, the system comprising:
a data input module configured to provide a starting enzyme structure comprising an enzyme scaffold library;
a similarity-search module configured to perform a similarity search against one or more known enzyme databases to characterize each enzyme scaffold with respect to one or more features selected from the group consisting of enzyme domains, active-site pockets, coordination spheres, immunogenicity profiles, stability, and kinetic parameters;
an optimization module suite comprising a plurality of optimization modules configured to generate engineered enzyme variants based on the characterized enzyme scaffolds;
an evaluation module configured to evaluate the engineered enzyme variants generated by the optimization modules according to predefined optimization goals and to compute a multi-property optimization (MPO) score;
a feedback-generation module configured to generate a feedback signal based on the MPO score and to iteratively refine parameters of the optimization modules to improve enzyme variant properties; and
an output module configured to produce optimized enzyme sequences in accordance with the predefined optimization goals.

15. The computer-implemented system of claim 14, wherein the optimization module suite comprising a plurality of optimization further comprises:
a cofactor binding optimization module configured to analyze and modify cofactor interaction regions of the enzyme scaffold;
a substrate binding optimization module configured to evaluate and optimize substrate-binding residues, pockets, and access tunnels;
a metal-ion coordination optimization module configured to assess and adjust parameters of the metal coordination environment to modulate catalytic function;
a hinge interaction optimization module configured to characterize and optimize hinge-region interactions to influence conformational transitions;
a surface and electrostatics optimization module configured to evaluate and refine surface charge distribution, solvent exposure, and electrostatic interactions; and
an allosteric network optimization module configured to identify and optimize long-range residue interaction networks that influence allosteric regulation.

16. The computer-implemented system of claim 14, further comprising a storage database configured to store the enzyme scaffold library, engineered variants, MPO evaluation results, and produced optimized enzyme sequences.

17. A non-transitory computer-readable medium storing instructions that, when executed by a computing device, cause the computing device to perform a method for preparing a framework for enzyme optimization, the method comprising:
providing a starting enzyme structure comprising an enzyme scaffold library;
performing a similarity search against one or more known enzyme databases to characterize each enzyme scaffold with respect to one or more features selected from the group consisting of enzyme domains, active-site pockets, coordination spheres, immunogenicity profiles, stability, and kinetic parameters;
executing a plurality of optimization modules to generate engineered enzyme variants;
evaluating the engineered enzyme variants generated by the optimization modules according to predefined optimization goals to compute a multi-property optimization (MPO) score;
generating a feedback signal based on the MPO score to iteratively refine the optimization modules and improve enzyme variant properties; and
producing optimized enzyme sequences in accordance with the predefined optimization goals.

18. The non-transitory computer-readable medium of claim 17, wherein the instructions further cause the computing device to perform sequence-or structure-based similarity searches using alignment algorithms.

19. The non-transitory computer-readable medium of claim 17, wherein the plurality of optimization modules comprises at least one of: cofactor binding optimization, substrate binding optimization, metal-ion coordination optimization, structural stability optimization, kinetic optimization, and immunogenicity reduction.

20. The non-transitory computer-readable medium of claim 17, wherein evaluating the engineered enzyme variants comprises computing the MPO score as a weighted combination of catalytic, structural, and immunological performance metrics.
